(19) United States
(12) Patent Application Publication
(10) Pub. No.: US 2009/0029907 A1
(43) Pub. Date: Jan. 29, 2009

(54) Recombinant Method for Production of an Erythropoiesis Stimulating Protein

(75) Inventor: Villoo Morawala Patell, Bangalore (IN)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: AVESTHAGEN LIMITED, Bangalore (IN)

(21) Appl. No.: 11/914,518

(22) PCT Filed: May 24, 2006
(86) PCT No.: PCT/IB2006/001353

§ 371 Date: Nov. 15, 2007

(30) Foreign Application Priority Data
May 24, 2005 (IN) 627/CHE/2005

(51) Int. Cl. A61K 38/22; C12P 21/02

(52) U.S. Cl. 514/8; 435/69.4

(57) ABSTRACT

The present invention relates to the recombinant method used for the production of a highly glycosylated form (in total five N linked glycosylations as opposed to three N linked glyosylations in the natural EPO) of erythropoietin. The added sites for glycosylation will result in greater number of carbohydrate chains, and higher sialic acid content than human EPO, which in turn would impart to the recombinant molecule a longer half-life. The invention further relates to the construction of expression cassettes comprising nucleic acid sequences encoding for the highly glycosylated form of Erythropoietin and stable expression in the host cells. The invention further relates to the optimized method for purification of the erythropoiesis stimulating protein. The recombinant EPO according to the invention, and the salts and functional derivatives thereof, may comprise the active ingredient of pharmaceutical compositions for an increase in the hematocrit for treatment of anemia and for restoration of patient well being and quality of life.

### FIELD OF INVENTION

The present invention relates to the recombinant method used for the production of a highly glycosylated form (in total five N linked glycosylations as opposed to three N linked glyosylations in the natural EPO) of erythropoietin. The added sites for glycosylation will result in greater number of carbohydrate chains, and higher sialic acid content than human EPO, which in turn would impart to the recombinant molecule a longer half-life.

The invention further relates to the construction of expression cassettes comprising nucleic acid sequences encoding for the highly glycosylated form of Erythropoietin and stable expression in the host cells.

The invention further relates to the optimized method for purification of the erythropoiesis stimulating protein.

The recombinant EPO according to the invention, and the salts and functional derivatives thereof, may comprise the active ingredient of pharmaceutical compositions for an increase in the hematocrit for treatment of anemia and for restoration of patient well being and quality of life.

### BACKGROUND OF THE INVENTION

Erythropoietin (EPO) is a glycoprotein hormone that is the primary regulator of erythropoiesis or maintenance of the body's red blood cell mass at an optimum level. In response to a decrease in tissue oxygenation, EPO synthesis increases in the kidney. The secreted hormone bind specific receptors on the surface of red blood cell precursors in the bone marrow, leading to their survival, proliferation, differentiation and ultimately to an increase in the haematocrit (the ratio of the volume occupied by packed red blood cells to the volume of the whole blood).

Since its introduction more than a decade ago, recombinant human EPO (rHuEPO) has become the standard of care in treating the anemia associated with chronic renal failure (CRF). It is highly effective in correcting the anemia, restoring energy levels, and increasing patient well being and quality of life. It has also been approved for the treatment of anemia associated with cancer, HIV infection, and use in surgical setting to decrease the need for allogenic blood transfusions.

The recommended and usual therapy with rHuEPO is two to three times per week by subcutaneous or intravenous injection. For CRF patients, the duration of therapy is the life for the life of the patient, or until a successful kidney transplant restores kidney function, including the production of the hormone. For cancer patients, rHuEPO therapy is indicated for as long as the anemia persists, generally through the entire course of chemotherapy. However, the bioavailability of commercially available protein therapeutics such as EPO is limited by their short plasma half-life and susceptibility to protease degradation.

Thus it is an object of the present invention to provide recombinant method used for the production of separate and isolated isoforms of erythropoietin having a defined sialic acid content, longer half life and thus increased biological activity.

### SUMMARY OF THE INVENTION

The present invention relates to the recombinant method used for the production of a highly glycosylated form (in total five N linked glycosylations as opposed to three N linked glyosylations in the natural EPO) of erythropoietin. The added sites for glycosylation will result in greater number of carbohydrate chains, and higher sialic acid content than human EPO, which in turn might impart to the recombinant molecule a longer half-life.

Also provide by the present invention are novel biologically functional vital and circular plasmid DNA vectors incorporating DNA sequences of the invention and host organisms stably transformed or transfected with said vectors.

Correspondingly provided by the invention are novel methods for the production of useful polypeptides comprising cultured growth of such transformed or transfected hosts under conditions facilitative of large scale expression of the exogenous, vector-borne DNA-sequences and isolation of the desired polypeptides from the growth medium, cellular lysates or cellular membrane fractions.

One aspect of the invention pertains to the construction of expression cassettes comprising nucleic acid sequences encoding for the highly glycosylated form of Erythropoietin.

Compared to unmodified EPO and conventional EPO-PEG conjugates, the protein of the present invention has an increased circulating half-life and plasma residence time, decreased clearance, and increased clinical activity in vivo. The recombinant EPO according to the invention, and the salts and functional derivatives thereof, may comprise the active ingredient of pharmaceutical compositions for an increase in the hematocrit value for treatment of anemia and for restoration of patient well being and quality of life.

Numerous aspects and advantages of the invention will be apparent to those skilled in the art upon consideration of the following detailed description, which provides illustrations of the practice of the invention in its presently preferred embodiments.

### DETAILED DESCRIPTION OF THE FIGURES AND SEQUENCES

SEQ ID. No. 1. Nucleotide sequence encoding the novel erythropoiesis stimulating protein

SEQ ID No. 2. Codon-optimized version of the nucleotide sequence encoding the novel erythropoiesis stimulating protein.

SEQ ID No. 3. Amino acid sequence of NESP or Darbepoietin alfa

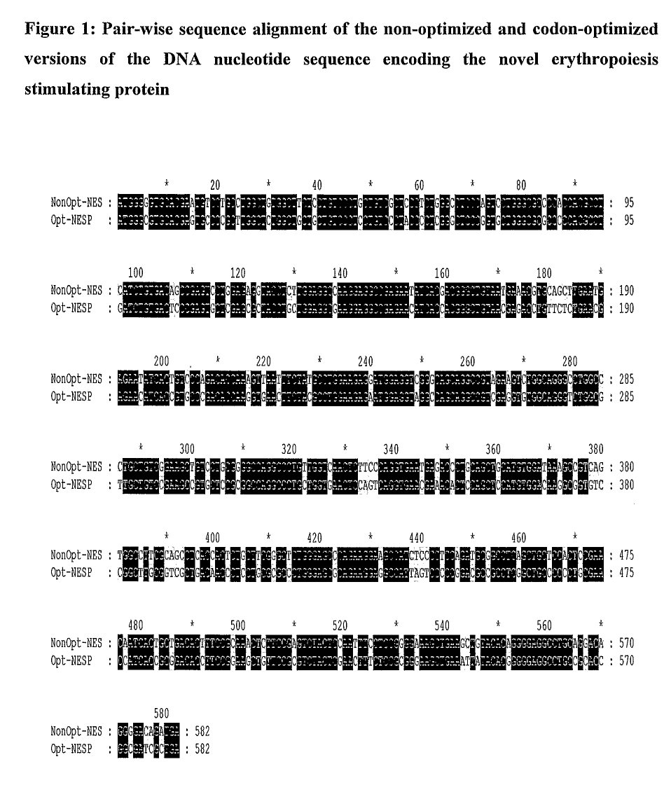

FIG. 1: Pair-wise sequence alignment of the non-optimized and codon-optimized versions of the DNA nucleotide sequence encoding the novel erythropoiesis stimulating protein

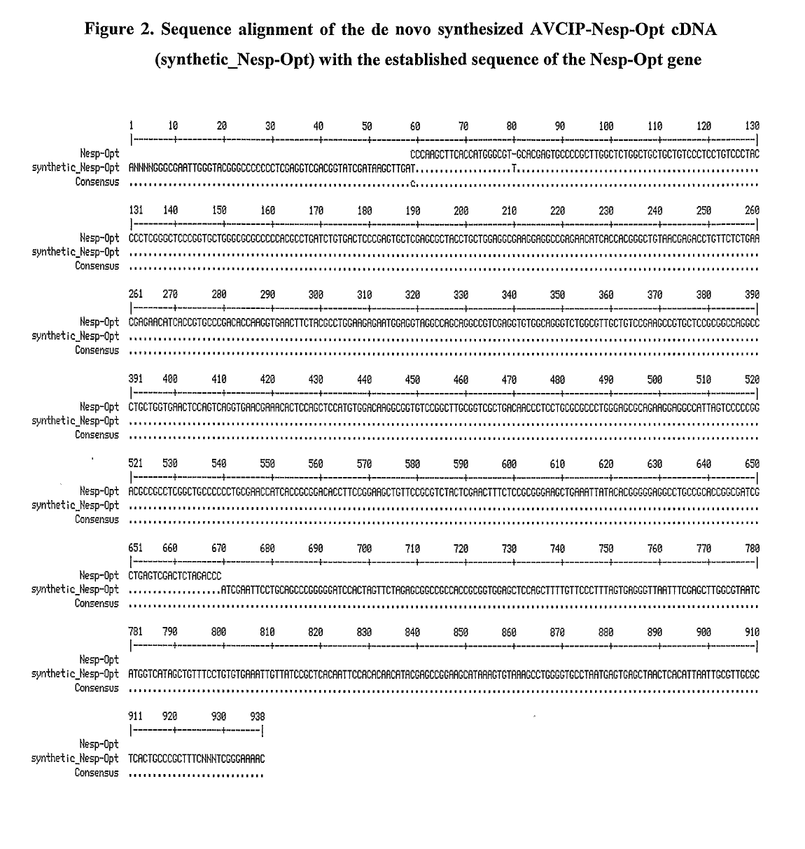

FIG. 2. Sequence alignment of the de novo synthesized optimized cDNA sequence of Erythropoiesis stimulating protein (AVCIP-Nesp-Opt) with the established and further optimized sequence of the Erythropoiesis stimulating protein (synthetic_Nesp-Opt)

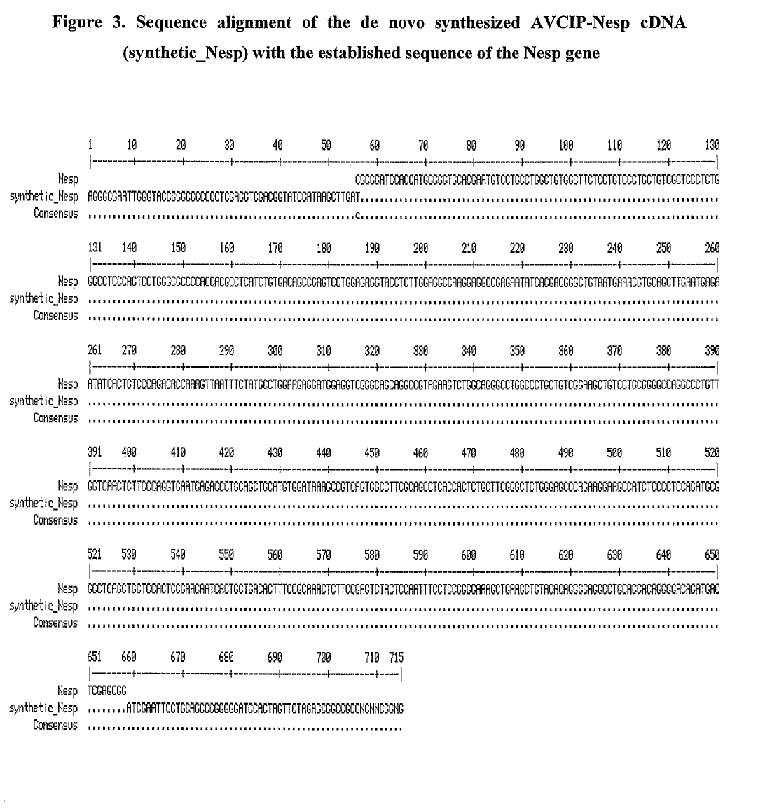

FIG. 3. Sequence alignment of the de novo synthesized cDNA sequence of Erythropoiesis stimulating protein (AVCIP-Nesp) with the established sequence of the Erythropoiesis stimulating protein (synthetic_Nesp)

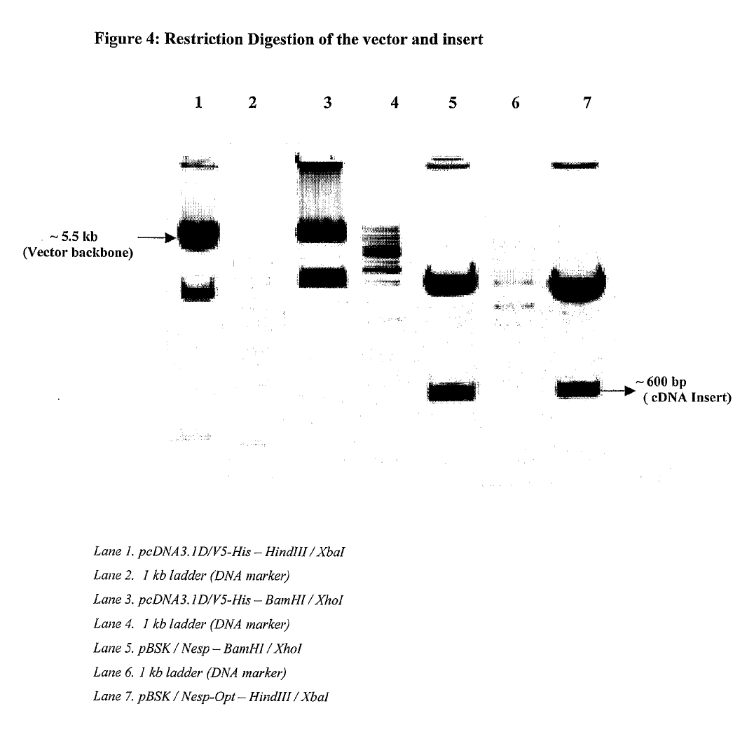

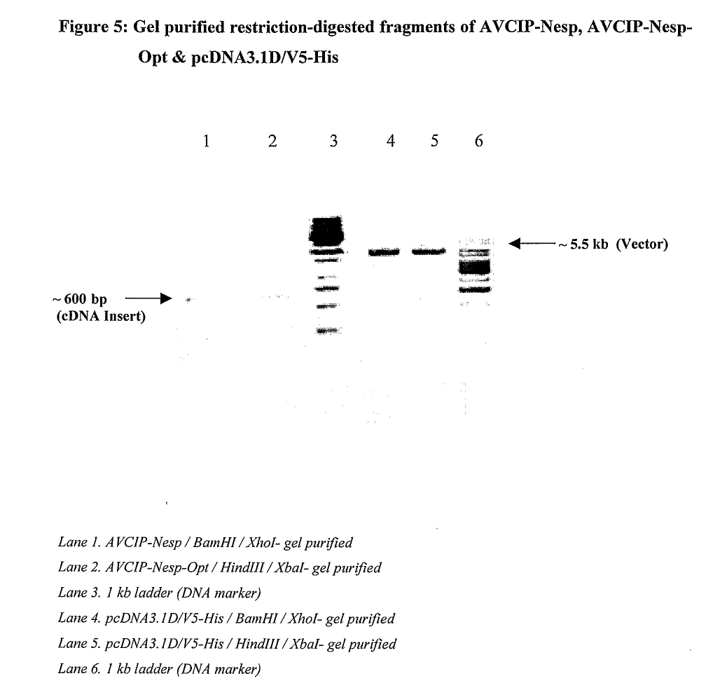

FIG. 4: Restriction Digestion of the vector and insert

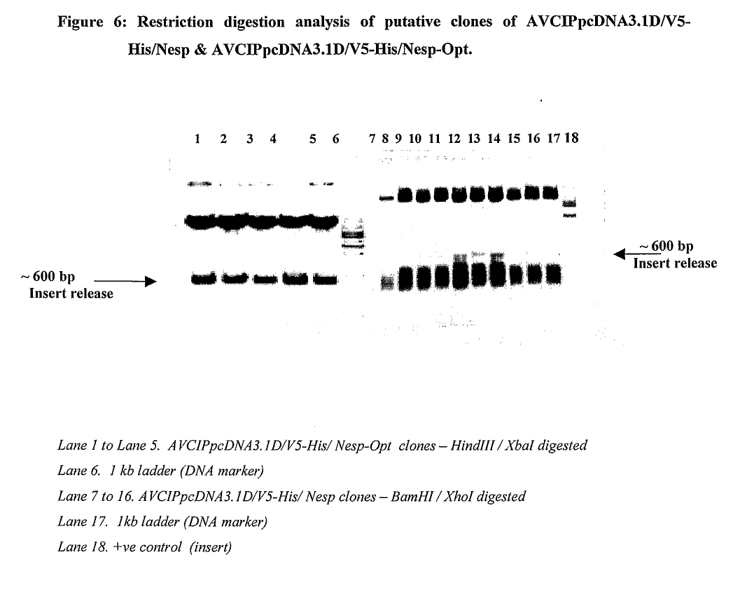

FIG. 5: Gel purified restriction-digested fragments of AVCIP-Nesp, AVCIP-Nesp-Opt & pcDNA3.1D/V5-His

FIG. 6: Restriction digestion analysis of putative clones of AVCIPpcDNA3.1D/V5-His/Nesp & AVCIPpcDNA3.1D/V5-His/Nesp-Opt.

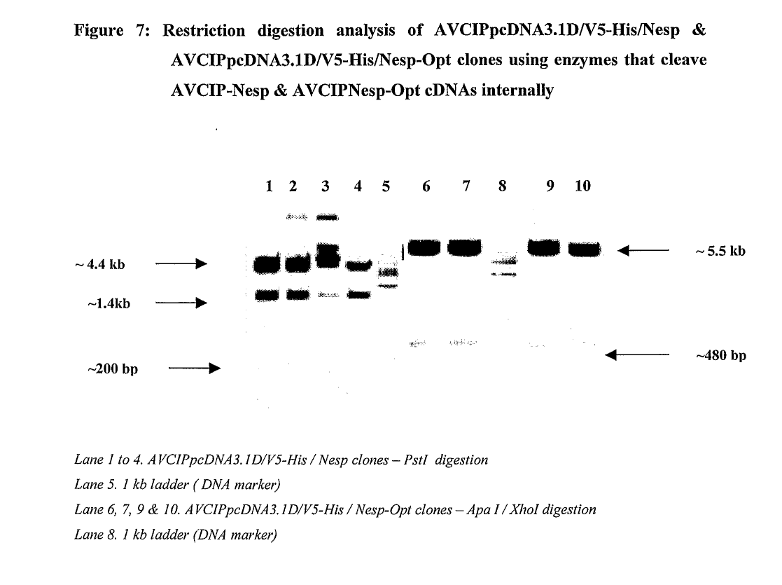

FIG. 7: Restriction digestion analysis of AVCIPpcDNA3.1D/V5-His/Nesp & AVCIPpcDNA3.1D/V5-His/Nesp-Opt clones using enzymes that cleave AVCIP-Nesp & AVCIPNesp-Opt cDNAs internally

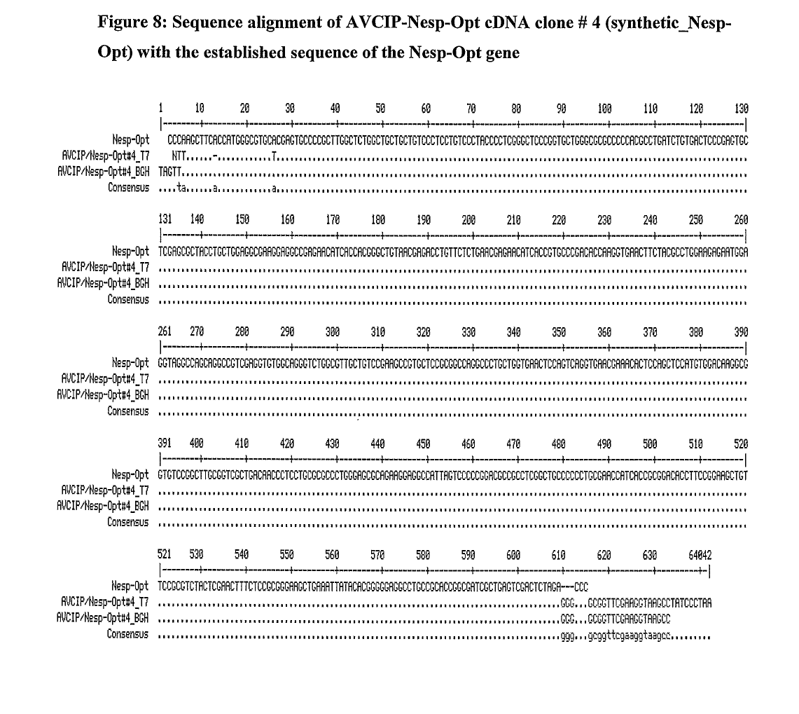

FIG. 8: Sequence alignment of AVCIP-Nesp-Opt cDNA clone # 4 (synthetic_Nesp-Opt) with the established sequence of the Nesp-Opt gene

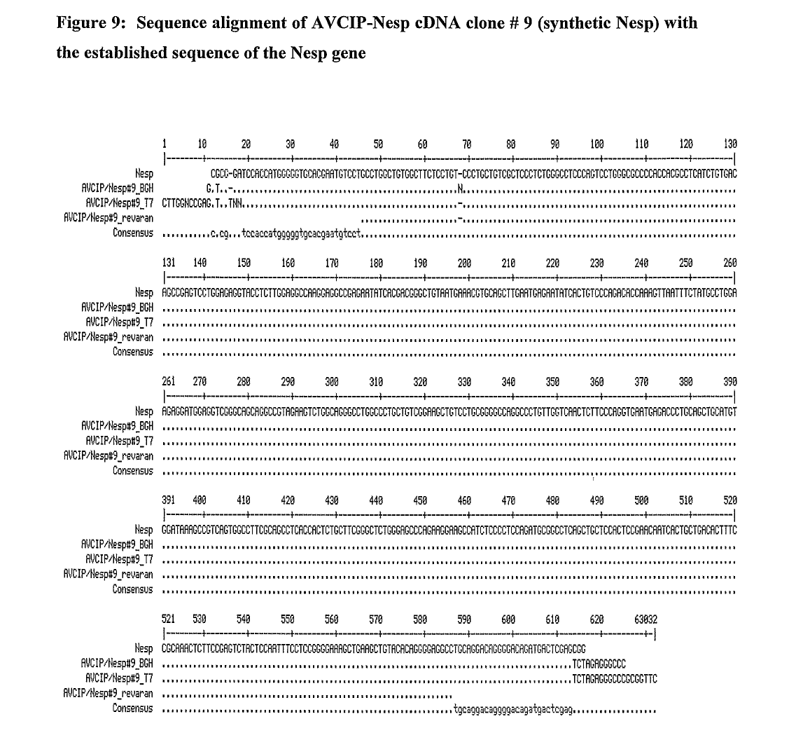

FIG. 9: Sequence alignment of AVCIP-Nesp cDNA clone # 9 (synthetic Nesp) with the established sequence of the Nesp gene

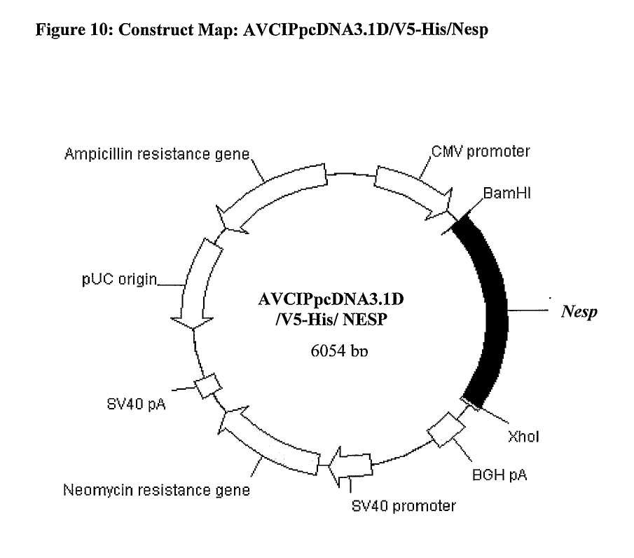

FIG. 10: Construct Map of AVCIPpcDNA3.1D/V5-His/Nesp

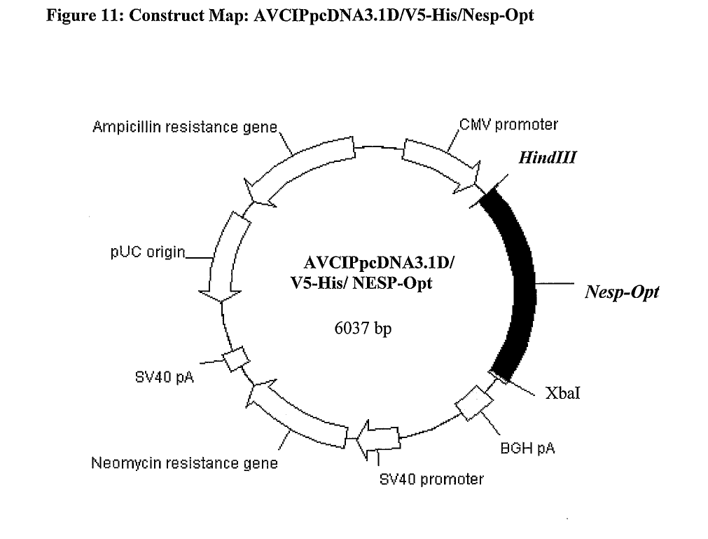

FIG. 11: Construct Map of AVCIPpcDNA3.1D/V5-His/Nesp-Opt

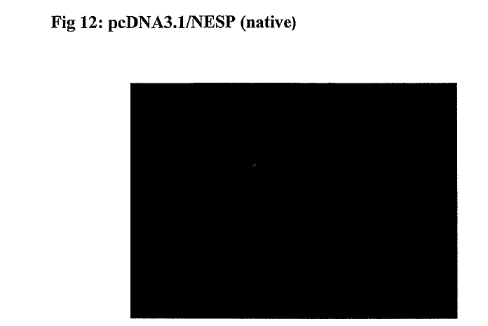

FIG. 12: pcDNA3.1/NESP (native)

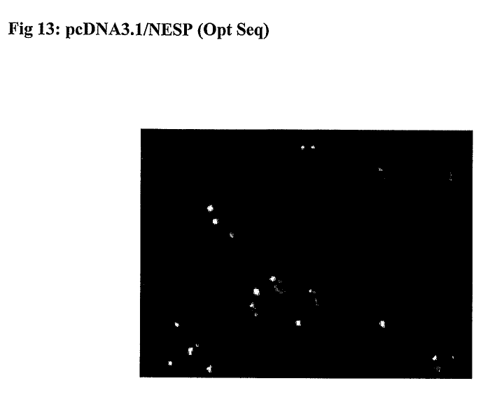

FIG. 13: pcDNA3.1/NESP (Opt Seq)

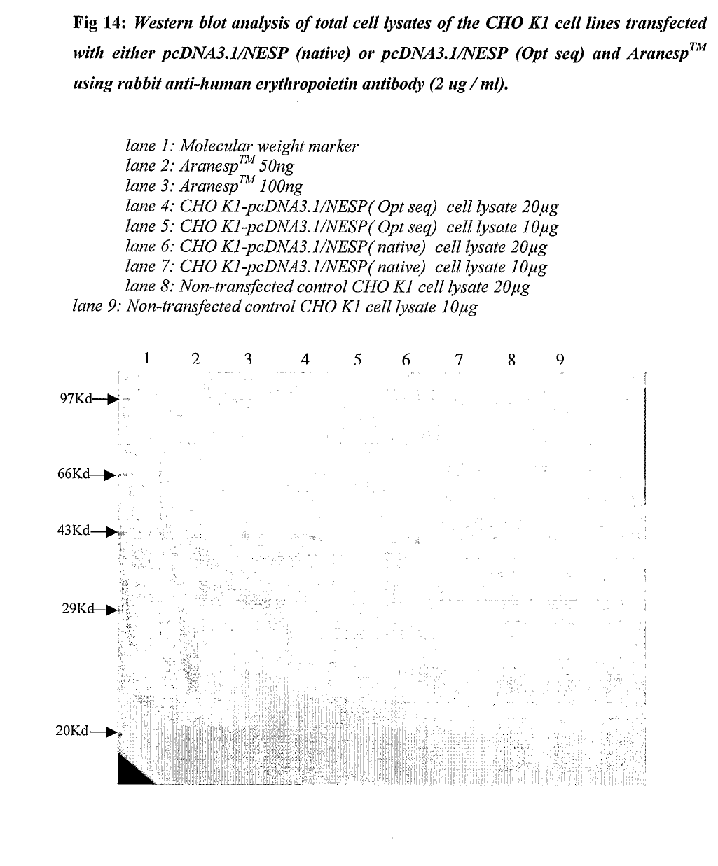

FIG. 14: Western blot analysis of total cell lysates of the CHO K1 cell lines transfected with either pcDNA3.1/NESP (native) or pcDNA3.1/NESP (Opt seq) and Aranesp™ using rabbit anti-human erythropoietin antibody (2 ug/ml).

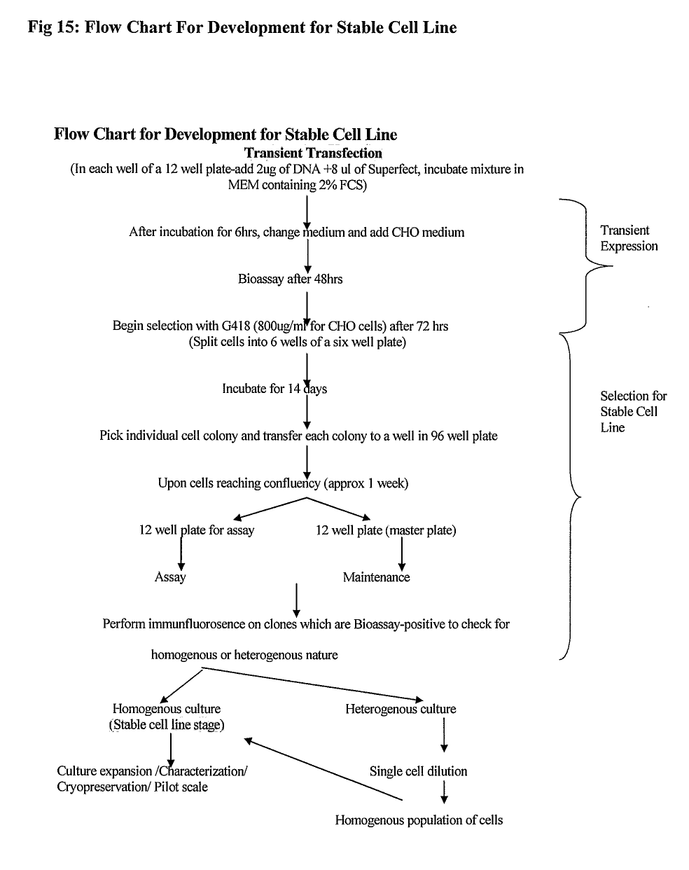

FIG. 15: Flow Chart For Development for Stable Cell Line

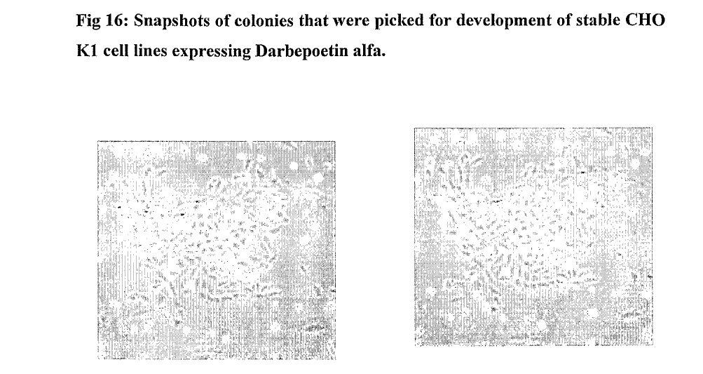

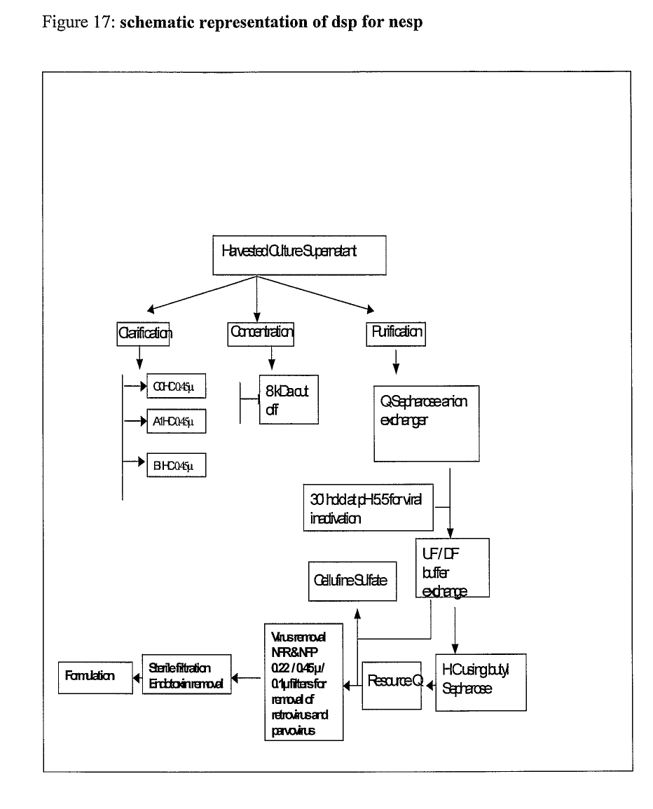

FIG. 16: Snapshots of colonies that were picked for development of stable CHO K1 cell lines expressing Darbepoetin alfa.

### DETAILED DESCRIPTION OF THE INVENTION

The subject invention provides alternative novel recombinant method for the production of erythropoietin isoforms. The specific isoforms of erythropoietin obtained in accordance with the present invention, and their properties, may vary depending upon the source of the starting material. In a preferred embodiment, the invention relates to an alternative novel recombinant method for the production of erythropoietin isoform, which differs, from recombinant human Erythropoietin (rHuEPO) and natural human EPO at five positions (Ala 30 Asn; His 32 Thr; Pro 87 Val; Trp 88 Asn and Pro 90 Thr).

The term “erythropoietin isoform” as used herein refers to erythropoietin preparations having a single isoelectric point (pI), and having the same amino acid sequence. The term “erythropoietin”, as used herein, includes naturally occurring erythropoietin, urinary derived human erythropoietin as well as non-naturally occurring polypeptides having an amino acid sequence and glycosylation sufficiently duplicative of that of naturally occurring erythropoietin to allow possession of in vivo biological properties of causing bone marrow cells to increase production of reticulocytes and red blood cells.

According to the present invention, DNA sequences encoding highly glycosylated form of human erythropoietin were synthesized by de novo approach. This approach would enable better codon optimization with respect to the particular mammalian cell to be used. Further the synthetic DNA was made the subject of eucaryotic/prokaryotic expression providing isolatable quantities of polypeptides displaying biological properties of naturally occurring Erythropoietin (EPO) as well as both in vivo and in vitro biological activities of EPO.

The following examples are presented by way of illustration of the invention and are specifically directed to procedures carried out prior to identification of EPO encoding monkey cDNA clones and human genomic clones, to procedures resulting in such identification, and to the sequencing, development of expression systems and immunological verification of EPO expression in such systems.

### EXAMPLE 1

### Synthesis of the Recombinant Erythropoiesis Stimulating Protein (NESP)

DNA sequences encoding highly glycosylated form of human erythropoietin were synthesized by de novo approach. This approach would enable better codon optimization with respect to the particular mammalian cell to be used. Further the synthetic DNA was made the subject of eucaryotic/prokaryotic expression providing isolatable quantities of polypeptides displaying biological properties of naturally occurring Erythropoietin (EPO) as well as both in vivo and in vitro biological activities of EPO.

Nucleotide sequence encoding the Erythropoiesis stimulating protein has been represented in the SEQ ID No. 1. The nucleotide residues that have been altered to incorporate additional glycosylation sites in said Erythopoiesis stimulating protein in comparison to the naturally occurring transcript of the human gene encoding erythropoietin have been highlighted in uppercase.

The codons in the coding region of Erythopoiesis stimulating protein have been altered as part of the codon optimization process to ensure optimal recombinant protein expression in mammalian cell lines such as CHO K1 and HEK 293. SEQ ID No. 2 represents codon optimized nucleotide sequence encoding Erythopoiesis stimulating protein.

Pair-wise sequence alignment of the non-optimised and codon optimized nucleotide sequence encoding Erythopoiesis stimulating protein has been represented in FIG. NO. 1.

SEQ ID. No. 3 depicts the complete primary amino acid sequence of Erythropoiesis stimulating protein of the invention. The amino acid residues of NESP that have been altered in comparison to the naturally occurring human EPO have been highlighted.

### EXAMPLE 2

### Verification of Authenticity of de novo Synthesized cDNA Encoding the Erythropoiesis Stimulating Protein

The verification of the authenticity of the de novo synthesized cDNA sequence original (AVCIP-Nesp) and codon optimized cDNA sequence (AVCIP-Nesp-Opt) was done by automated DNA sequencing and the results obtained are depicted in FIGS. No. 2 & 3.

### EXAMPLE 3

### Sub-Cloning of AVCIP-Nesp & AVCIP-Nesp-Opt cDNAs into the pcDNA3.1D/V5-His Mammalian Cell-Specific Expression Vector

The de novo synthesized cDNA sequence original (AVCIP-Nesp) and codon optimized cDNA sequence (AVCIP-Nesp-Opt) were individually sub-cloned into the mammalian cell-specific expression vector pcDNA3.1D/V5-His to generate the transfection-ready constructs. The details of the procedures used are given below:

### A. Reagents and Enzymes:

1. QIAGEN gel extraction kit & PCR purification kit

2. pcDNA 3.1D/V5-His vector DNA (Invitrogen)

| Enzyme | U/μl | 10x buffer |
|---|---|---|
| 1. BamHI | 10 | Buffer E |
| 2. XhoI | 10 | Buffer E |
| 3. HindIII | 20 | Buffer C |
| 4. XbaI | 10 | Buffer C |
| 5. T4 DNA ligase | 40 | Ligase Buffer |

All reactions were carried out as recommended by the manufacturer. For each reaction the supplied 10× reaction buffer was diluted to a final concentration of 1×.

### B. Restriction Digestion of the Vector and the Insert:

Procedure

The following DNA samples and restriction enzymes were used:

| DNA samples | Restriction Enzyme |
|---|---|
| Rxn # 1 Vector (for Nesp cloning) | BamHI/XhoI |
| Rxn # 2 Vector (for Nesp-Opt cloning) | HindIII/XbaI |
| Rxn # 3 pBSK/Nesp (2A) | BamHI/Xho I |
| Rxn # 4 pBSK/Nesp-Opt (2B) | HindIII/XbaI |

Restriction Enzyme Digest Reaction:

| Components | Final conc. | Rxn #1 | Rxn # 2 | Rxn # 3 | Rxn # 4 |
|---|---|---|---|---|---|
| Water | — | 2 μl | 2 μl | 2 μl | 2 μl |
| 10x Buffer | 1x | 2 μl | 2 μl | 3 μl | 3 μl |
| DNA | — | 12 μl | 12 μl | 20 μl | 20 μl |
| BamHI | 0.5U | 1 μl | — | 1 μl | — |
| XhoI | 0.5U | 1 μl | — | 1 μl | — |
| HindIII | 1.0U | — | 1 μl | — | 1 μl |
| XbaI | 0.5U | — | 1 μl | — | 1 μl |
| 10x BSA | 1x | 2 l | 2 μl | 3 μl | 3 μl |
| Final volume | 20 μl | 20 μl | 20 μl | 30 μl | 30 μl |

The reaction was mixed, spun down and incubated for 2 hrs at 37° C. The restriction digestion was analyzed by agarose gel electrophoresis. The expected digestion pattern was observed that featured a gene fragment fall out of ˜600 bp (for Rxn # 3 & 4) and a vector backbone fragment of ˜5.5 kb for Vector (Rxn # 1 & 2) was seen. (FIG. No. 4)

The ˜600 bp DNA fragments representing AVCIP-Nesp & AVCIP-Nesp-Opt cDNAs were separately purified by the gel extraction method using the QIAGEN gel extraction kit. The ˜5.5 kb digested vector backbone of the pcDNA3.1D/V5-His mammalian expression vector was also purified using the same kit.

Subsequent to the restriction digestion and gel-extraction of the requisite cDNA and vector DNA fragments, an aliquot (1-2 microliter) of each purified DNA sample was analyzed using agarose gel electrophoresis to check for purity and integrity as shown in FIG. No. 5.

C. Ligation of pcDNA3.1D/V5-His Backbone With AVCIP-Nesp & AVCIP-Opt-Nesp cDNAs:

The DNA concentration of the digested & purified vector and insert fragments was estimated and ligation was set up in the following manner:

| | | Rxn #1 | Rxn # 2 |
|---|---|---|---|
| Components | Final conc. | (AVCIP/Nesp) | (AVCIP-Nesp-Opt) |
| Water | — | 5 μl | 5 μl |
| 10xRxn buffer | 1x | 2 μl | 2 μl |
| Vector | ~50 ng | 2 μl | 2 μl |
| Insert | ~17 ng | 1 μl | 1 μl |
| T4 DNA ligase | 40U | 1 μl | 1 μl |
| Final volume | 10 μl | 10 μl | 10 μl |

The reactions were gently mixed, spun down and incubated at R.T, 2-3 hrs. JM109 competent cells were transformed with the contents of ligation reaction mixtures.

### D. Restriction Digestion Analysis of Putative Clones of AVCIPpcDNA3.1D/V5-His/Nesp & AVCIPpcDNA3.1D/V5-His/Nesp-Opt.

Plasmid DNA was individually purified from the colonies obtained on L.B agar plates containing ampicillin and the presence of the desired cDNA insert was confirmed by restriction digestion analysis of the isolated plasmid DNA as shown in FIG. No. 6.

In accordance with the results obtained after the restriction digestion of several putative clones containing the AVCIPpcDNA3.1D/V5-His/Nesp & AVCIPpcDNA3.1D/V5-His/Nesp-Opt, some of the clones which showed the desired restriction pattern were selected for further restriction digestion analysis using restriction enzymes that cleave the AVCIP-Nesp & AVCIPNesp-Opt cDNAs internally to generate variable sized fragments as shown below in FIG. No. 7.

### E. Verification of Selected Clones of AVCIPpcDNA3.1D/V5-His/Nesp & AVCIPpcDNA3.1D/V5-His/Nesp-Opt by DNA Sequencing

The AVCIPpcDNA3.1D/V5-His/Nesp & AVCIPpcDNA3.1D/V5-His/Nesp-Opt clones selected as a result of the restriction mapping analysis were further verified by automated DNA sequencing.

| NOMENCLATURE | DESCRIPTION OF PRIMERS | SEQUENCES |
|---|---|---|
| T7 Sequencing primer | Invitrogen kit primer | 5′ TAATACGACTCACTATAGGG 3′ |

AVCIPpcDNA3.1D/V5-His/Nesp & AVCIPpcDNA3.1D/V5-His/Nesp-Opt clones showed 100% identity with the template sequence, as shown in FIGS. No. 8 & 9.

The maps of the recombinant expression constructs made using the de novo synthesized AVCIP-Nesp and AVCIP-Nesp-Opt cDNAs are pictorially represented in the FIGS. No. 10 & 11.

### EXAMPLE 4

### Maintenance and Propagation of the cDNA Fusion Construct

The maintenance and propagation of the cDNA construct encoding the novel erythropoiesis stimulating protein was carried out in a standard bacterial cell line such as Top 10 (Invitrogen).

### EXAMPLE 5

### Transient/Stable Recombinant Protein Expression in CHO-K1 C Cells

### (a) Transient Expression of Erythropoiesis Stimulating Protein in CHO K1 Cells:

The optimized protocol for transfection of plasmid DNA was used to transfect CHO cells with:
1. pcDNA3.1/NESP (native)
2. pcDNA3.1/NESP (Opt seq)

### Transient Transfection of Adherent CHO K1 Cells

1. The day before transfection, seed 1×10⁵ cells per well in a 24 well plate in 1 ml growth medium (D-MEM/F 1:1). The cell number seeded should produce 80% confluence on the day of transfection.

2. Incubate the cells under their normal growth conditions (generally 37° C. and 5% CO2).

3. On the day of transfection, Tube A—dilute 2 μg DNA dissolved in TE buffer pH 7.0 to pH 8.0 (minimum DNA concentration: 0.1 μg/μl) with Opti-MEM™ to a total volume of 100 μl. Mix and spin down the solution for a few seconds to remove drops from the top of the tube.

4. Tube B-Add 6 μl Lipofectamine™ 2000 transfection Reagent in 100 μl of Opti-MEM™ and allow to stand at room temperature for 5 minutes.

5. Mix contents of Tube A and Tube B by pipetting up and down 5 times.

6. Incubate the samples for 15 min at room temperature (15-25° C.) to allow transfection-complex formation.

7. While complex formation takes place, gently aspirate the growth medium from the dish, and wash cells once with 2 ml PBS.

8. Add 0.1 ml cell Opti-MEM™ to the reaction tube containing the transfection complexes. Mix by pipetting up and down twice, and immediately transfer the total volume to the cells in the one well of a 24 well plate.

9. Incubate cells with the transfection complexes for 6 hours under their normal growth conditions.

10. Remove medium containing the remaining complexes from the cells by gentle aspiration, and wash cells once with 4 ml PBS (phosphate buffered saline).

11. Add fresh cell growth medium (containing serum and antibiotics). Assay cells for expression of the transfected gene after an appropriate incubation time.

These transfected cells were stained with anti-erythropoietin antibody to evaluate the expression of the protein. As depicted in FIGS. No. 12 & 13, specific expression of the said protein was detected in both sets of transient transfection experiments representing CHO K1 cell lines independently transfected with pcDNA3.1/NESP (native) and pcDNA3.1/NESP (Opt seq).

### (b). Detection of Transient Expression of Erythropoiesis Stimulating Protein in Transfected CHO K1 Cell Lines by Western Blotting.

Total cell lysates were prepared from CHO K1 cell lines that were independently transfected with either pcDNA3.1/NESP (native) or pcDNA3.1/NESP(Opt Seq). The said cell lysates were prepared 48 hrs after the transfection event and two different amounts of the total protein preparation (10 □g and 20 □g) of the cell lysates were electrophoresed on a 12% SDS-PAGE prior to blotting on to a PVDF membrane. The PVDF membrane was then probed with 2 □g/ml of rabbit anti-human erythropoietin antibody and the result obtained is shown in FIG. No. 14.

As evident from FIG. 8, the presence of Erythropoiesis Stimulating Protein was specifically detected in the total cell lysates of the CHO K1 cell lines transfected with either pcDNA3.1/NESP (native) or pcDNA3.1/NESP(Opt Seq) at higher concentrations (˜20 □g) of the protein preparations used. The electrophoretic mobility of said Erythropoiesis Stimulating Protein present in the cell lysates of the transfected CHO K1 cell lines was found to match that observed in the case of the therapeutic formulation, Aranesp™, thereby indicating the expected hyper-glycosylated nature of the expressed recombinant protein.

### (c) Development of Stable CHO K1 Cell Lines Expressing Erythropoiesis Stimulating Protein

Integration of DNA into the chromosome, or stable episomal maintenance, of reporter genes and other genes has been known to occur with a relatively low frequency. The ability to select for these cells is made possible using genes that encode resistance to a lethal drug. An example of such a combination is the marker gene for neomycin phosphotransferase with the drug Geneticin™. Individual cells that survive the drug treatment expand into clonal groups that can be individually selected, propagated and analyzed. A flow chart depicting the steps involved in the development of stable line is shown in FIG. No. 15.

### Protocol 2: Stable Transfection of Adherent CHO K1 Cells.

1. The day before transfection, seed 1×10⁵ cells per well in a 24 well plate in 1 ml growth medium (D-MEM/F 1:1). The cell number seeded should produce 80% confluence on the day of transfection.

2. Incubate the cells under their normal growth conditions (generally 37° C. and 5% CO2).

3. On the day of transfection, Tube A—dilute 2 μg DNA dissolved in TE buffer pH 7 to pH 8 (minimum DNA concentration: 0.1 μg/μl) with Opti-MEM to a total volume of 100 μl. Mix and spin down the solution for a few seconds to remove drops from the top of the tube.

4. Tube B-Add 6 μl Lipofectamine 2000 transfection Reagent in 100 μl of Opti-MEM and allow to stand at room temperature for 5 minutes.

5. Mix contents of Tube A and Tube B by pipetting up and down 5 times.

6. Incubate the samples for 15 min at room temperature (15-25° C.) to allow transfection-complex formation.

7. While complex formation takes place, gently aspirate the growth medium from the dish, and wash cells once with 2 ml PBS.

8. Add 0.1 ml cell Opti-MEM to the reaction tube containing the transfection complexes. Mix by pipetting up and down twice, and immediately transfer the total volume to the cells in the one well of a 24 well plate.

9. Incubate cells with the transfection complexes for 6 hours under their normal growth conditions.

10. Remove medium containing the remaining complexes from the cells by gentle aspiration, and wash cells once with 4 ml PBS.

11. Add fresh cell growth medium (containing serum and antibiotics). Assay cells for expression of the transfected gene after an appropriate incubation time.

12. Passage cells at 1:10 to 1:15 into the appropriate selective medium. Maintain cells in selective medium under their normal growth conditions until colonies appear.

### EXAMPLE 6

### Selection of Stable CHO K1 Cell Lines Expressing Erythropoiesis Stimulating Protein

Transiently expressing CHO cells transfected with either pcDNA3.1/NESP(native), pcDNA3.1/NESP(Opt Seq) were trypsinized and diluted in selection medium containing 1 mg/ml of Geneticin™. The cells were incubated for 14 days in selection medium until colonies could be isolated (FIGS. 2A & B below). In all, 89 of pcDNA3.1 NESP (native) and 91 colonies pcDNA3.1 NESP(Opt-Seq) were picked up in sterile condition and plated in single well per colony of a 96 well plate.

Avesthagen has selected 89 colonies of CHO K1/pcDNA3.1/NESP (native) and 91 colonies of CHO/pcDNA3.1/-NESP (Opt-seq) in order to develop producer cell lines over-expressing Erythropoiesis Stimulating Protein. All the CHO K1 cell colonies selected thus far will be analyzed by immunofluorescence, Western blotting, ELISA and cell-based functional assays so as to generate a single cell-derived CHO K1 producer cell line stably expressing Erythropoiesis Stimulating Protein of the said invention.

### EXAMPLE 7

### Purification of Novel Erythropoiesis Stimulating Protein

The quality and bio-safety of a biopharmaceutical is, to a great extent, dependent on the extraction procedures used to manufacture the purified product. On the one hand, downstream processing has to ensure an effective and economic isolation of the desired product from the culture broth or cellular material obtained during the cell culture process. However, on the other hand, components that would contaminate the final product must be reliably separated. Different types of components that should not be present in the final product formulation have to be removed during these steps.

The first group comprises media derived or process-derived impurities that can be of aproteinaceous or non-proteinaceous nature (e.g. lipids, antifoaming agents, antibiotics). This group also includes host-cell-derived impurities such as proteins, which might induce unwanted immune responses, or nucleic acids, which are a major concern because they might harbor potentially harmful genetic information when incorporated within healthy human cells. The second group consists of adventitious agents and contaminants and comprises viruses, virus-like particles (VLPs), bacteria, fungi, mycoplasmas and so on.

The removal of medium components and proteinaceous impurities is an integral part of product isolation. Procedures aimed at the removal of medium supplements, such as antibiotics or cytotoxic substances (e.g. geniticine or methotrexate) will be built into the purification strategy and appropriate tests will be established to validate their efficiency. Some impurities, such as DNA, can be reduced by a careful choice of cultivation and harvesting conditions. For practical reasons, it is not possible to manufacture a 100% pure product, acceptable concentration levels for the presence of impurities in the final product formulation have been defined. For example, the World Health Organization (WHO) defined the maximal acceptable amount of DNA to be 100 pg per single dose of a biotechnologically derived protein drug. The potential for inactivating adventitious agents during the purification step can be exploited or additional inactivation steps can be included, within the purification strategy. Viruses and VLPs, for example, can be inactivated by the application of inactivating chemicals (e.g. N-acetylethyleneimine, Tri-N-butylphosphate)¹⁰, organic solvents, chaotropic salts, extreme pH-values, irradiation, and so on. Temperature treatment achieved by the application of microwave technology has also been shown to inactivate viruses. Notwithstanding the above, the potential of the chosen technology for inactivation remains to be validated and this validation has also to prove that the inactivation method does not harm the product integrity.

Mature human EPO protein is comprised of an invariant sequence of 165 amino acids, which is derived from a 193 amino acid precursor in two steps. The N-terminal 27 amino acid leader sequence is cleaved off prior to the secretion of the hormone and the C-terminal Arg is proteolytically removed by an endogenous carboxypeptidase. Subsequent to the establishment of a contaminant-free cell culture system as per the guidelines of the regulatory agencies, that over-expresses the desired recombinant protein, the purification of novel erythropoiesis stimulating protein protein can be done using a series of steps involving dialysis-filtration and column chromatography procedures involving anion-exchange and reverse-phase matrices. The fractions containing the most highly branched glycans and highest sialic acid content will be recovered to maximize in vivo activity.

### EXAMPLE 8

### Optimization of Purification Procedures

Subsequent to the establishment of reproducible bioactivity in accordance with the recommended functional/binding assays mentioned above, efforts will be made to optimize the purification procedures so as to maximize the yield of recombinant NESP from stable, high-expressing cell line. Purification strategies will aim at process economics, speed to market, scalability, reproducibility, and maximum purity of the product with functional stability and structural integrity as the major objectives. To this effect, a combinatorial approach with both filtration (normal and tangential flow filtration) and chromatography would be explored. The process qualification requirements and acceptance criteria studies will be conducted on 3 batches.

Protein purification selectively utilizing the glycan component of a glycoprotein as a capture target is commonly performed using affinity chromatography. The most common matrices are m-aminophenylboronic acid agarose and the immobilized lectins, Concanavailn A Sepharose (Con A Sepharose) and wheat germ agglutinin Sepharose (WGA-Sepharose). Of the above-mentioned, m-aminophenylboronic acid matrices are capable of forming temporary bonds with any molecule containing a 1,2-cis-diol group while Con A matrices bind specifically to mannosyl and glucosyl residues containing unmodified hydroxyl groups at the C3, C4 and C6 positions. WGA Sepharose matrices are highly specific to N-acetyl glucosamine (NAG) or N-acetyl neuraminic acid (NANA or sialic acid) residues of the glycoprotein.

Accordingly, the purification process would comprise of the following downstream train:

a. Initial clarification and concentration using normal and tangential flow filtration procedures

b. Ultra filtration/Dialysis filtration (based on tangential flow filtration)

c. Chromo step—I: Affinity chromatography using lectin/m-amino phenyl based matrices. M-amino phenyl ligand based affinity medium would be more preferred.

d. Chromo step—II: Ion-exchange chromatography (IEX) using Q-Sepharose anion exchanger

e. Chromo step—III: Hydrophobic interaction chromatography (HIC) using butyl-Sepharose

f. Virus removal and sterile filtration

g. Endotoxin removal

h. Formulation.

Note: The sequence of unit operations during the chromo steps may be altered for high purity and maximum product recovery. The outcome of the purification process at each step will be evaluated for structural and functional integrity of the protein using physicochemical and immunological methods.

In another preferred embodiment, the purification process would aim at direct capture of the target protein from crude culture broth using anion exchange resin in the expanded bed adsorption mode as against conventional packed bed mode and would comprise of the following steps:

a. Anion exchange chromatography using Q-Sepharose XL by salt step elution as capture step.

b. Hydrophobic interaction chromatography (HIC) using butyl Sepharose

c. A second anion exchange chromatography using Resource Q as a polishing step

d. Virus removal and sterile filtration

e. Endotoxin removal

f. Formulation.

More preferably, a two-step purification process using anion exchange chromatography and HIC would be employed as the major chromatography steps depending on the % product recovery and purity. Subsequent steps as outlined in the above mentioned strategies would then follow.

Note: An optional acid wash step may be incorporated post anion exchange capture in both the strategies outlined above, depending on the capture efficiency for selective enrichment of isoforms of acidic pI with high glycosyl and sialyl contents and for the removal of contaminating unrelated basic proteins. Additionally, flow through based anion exchangers such as cellufine sulfate will be used for selective binding of process contaminants, endogenous/adventitious viruses and column extractables.

### EXAMPLE 9

### Establishment of the Identity of the Target Protein Using Biochemical, Immunological and Physico-Chemical Methods:

The percent recovery of the total protein at each stage will be quantitated using bicinchoninic acid procedure (BCA)/Bradford dye binding method. The target protein concentration at each stage of purification will be probed using highly specific and reliable enzyme based immunoassays such as direct or indirect sandwich ELISA More preferably, a double antibody sandwich ELISA would be adapted for evaluating the target protein concentrations. As NESP is a glycoprotein, a qualitative evaluation of the degree of glycosylation will be examined using specific staining procedures for glycoprotein detection of the electrophoresed SDS gels under reducing conditions. Qualitative and target specific western analysis will be followed at each stage. Reversed phase chromatography, isoelectric focusing and two-dimensional gel electrophoresis will be employed to evaluate the purified product. Secondary structural analysis would be examined using far UV circular dichroism. Molecular mass and oligomeric status will be investigated using size exclusion and MALDI-TOF. The investigations will also focus on the stability of the protein in relation to pH and temperature. As NESP is a hyperglycosylated protein, glycosylation patterns of the purified protein would be documented using gas chromatography (GC) analysis.

### EXAMPLE 10

### Assays for in vitro and in vivo Activity of Novel Erythropoiesis Stimulating Protein

Bioassays for detecting in vitro EPO-receptor binding of novel erythropoiesis stimulating protein will be done using:

(a) Competitive binding using I¹²⁵ labeled novel erythropoiesis stimulating protein

(b) [H]³-thymidine uptake using a recommended human cell line such as Ut7/EPO.

Pre-clinical in vivo bioactivity (normal haematocrit restoration ability) of novel erythropoiesis stimulating protein will be tested on recommended mouse lines such as BDF1.

1. A process for the preparation of an in vivo biologically active Erythropoiesis Stimulating Protein, comprising the steps of:
(a) growing, under suitable nutrient conditions, host cells transformed or transfected with an isolated DNA sequence selected from the group consisting of (i) the DNA sequences set out in SEQ ID No.1 and SEQ ID No. 2, (ii) the protein coding sequence represented in SEQ ID No. 3, and (iii) DNA sequences which hybridize under stringent conditions to the DNA sequences defined in (i) and (ii) or their complementary strands; and
(b) isolating said Erythropoiesis Stimulating Protein therefrom.

2. A process for the preparation of an in vivo biologically active erythropoietin product comprising the steps of transforming a host cell with a synthesized DNA sequence encoding Erythropoietin amino acid sequence of SEQ ID. 3 and isolating said erythropoietin product from said host cell or the medium of its growth.

3. The process according to claim 1 wherein said host cells are mammalian cells.

4. The process according to claim 1 wherein said host cells are CHO K1 cells.

5. A process for the production of a glycosylated erythropoietin polypeptide having the in vivo biological property of causing bone marrow cells to increase production of reticulocytes and red blood cells comprising the steps of:
a) growing, under suitable nutrient conditions, mammalian cells comprising promoter DNA, other than human erythropoietin promoter DNA, operatively linked to DNA encoding the mature erythropoietin amino acid sequence of SEQ ID No. 3; and
b) isolating said glycosylated erythropoietin polypeptide expressed by said cells.

6. The process of claim 5 wherein said promoter DNA is a viral promoter DNA.

7. A process for the preparation of an in vivo biologically active erythropoietin product comprising the steps of transforming a host cell with a vector construct of FIG. No. 10 or 11 and isolating said erythropoietin product from said host cell or the medium of its growth.

8. The process of claim 7, wherein said vector is a mammalian cell specific expression vector as represented in FIG. 10 or 11.

9. A pharmaceutical composition comprising a therapeutically effective amount of human erythropoietin and a pharmaceutically acceptable diluent, adjuvant or carrier, wherein said erythropoietin is purified from mammalian cells grown in culture.

10. A method of raising and maintaining hematocrit in a mammal comprising administering a therapeutically effective amount of a hyperglycosylated analog of erythropoietin, wherein the analog is administered less frequently than an equivalent molar amount of recombinant human erythropoietin to obtain a comparable target hematocrit.

11. The process according to claim 2 wherein said host cells are mammalian cells.

12. The process according to claim 2 wherein said host cells are CHO K1 cells.


SEQUENCE LISTING (3 sequences)

SEQ ID NO: 1 - DNA, 582 bp, Human
atgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtaatgaaacgtgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccaggtgaatgagaccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagatga

SEQ ID NO: 2 - DNA, 582 bp, Human
atgggcgtgcacgagtgccccgcttggctctggctgctgctgtccctcctgtccctacccctcgggctcccggtgctgggcgcgcccccacgcctgatctgtgactcccgagtgctcgagcgctacctgctggaggcgaaggaggccgagaacatcaccacgggctgtaacgagacctgttctctgaacgagaacatcaccgtgcccgacaccaaggtgaacttctacgcctggaagagaatggaggtaggccagcaggccgtcgaggtgtggcagggtctggcgttgctgtccgaagccgtgctccgcggccaggccctgctggtgaactccagtcaggtgaacgaaacactccagctccatgtggacaaggcggtgtccggcttgcggtcgctgacaaccctcctgcgcgccctgggagcgcagaaggaggccattagtcccccggacgccgcctcggctgcccccctgcgaaccatcaccgcggacaccttccggaagctgttccgcgtctactcgaactttctccgcgggaagctgaaattatacacgggggaggcctgccgcaccggcgatcgctga

SEQ ID NO: 3 - protein, 193 aa, Human
MGVHECPAWLWLLLSLLSLPLGLPVLGAPPRLICDSRVLERYLLEAKEAENITTGCNETCSLNENITVPDTKVNFYAWKRMEVGQQAVEVWQGLALLSEAVLRGQALLVNSSQVNETLQLHVDKAVSGLRSLTTLLRALGAQKEAISPPDAASAAPLRTITADTFRKLFRVYSNFLRGKLKLYTGEACRTGDR